(19) United States
(12) Patent Application Publication
(10) Pub. No.: US 2009/0324752 A1
(43) Pub. Date: Dec. 31, 2009

(54) COSMETIC USE OF ACTIVE INGREDIENTS INCREASING THE PRODUCTION OF GROWTH FACTORS

(75) Inventors: Christelle Marie Suzanne Lasserre, Jersey City, NJ (US); Yannick Gérard Maestro, Martigues (FR)

Correspondence Address: YOUNG & THOMPSON, 209 Madison Street, Suite 500, Alexandria, VA, 22314, US

(73) Assignee: CHANEL PARFUMS BEAUTE, NEUILY SUR SEINE CEDEX (FR)

(21) Appl. No.: 12/304,637

(22) PCT Filed: Jun. 11, 2007
(86) PCT No.: PCT/EP2007/055714

§ 371 Date: Apr. 15, 2009

(30) Foreign Application Priority Data
Jun. 12, 2006 (FR) 06 05210
Jul. 19, 2006 (US) 60831667

(51) Int. Cl. A61K 36/00

(52) U.S. Cl. 424/725

(57) ABSTRACT

The invention relates to a cosmetic skin care method, intended to prevent and/or treat at least one cutaneous sign of ageing, including topical application to the skin of a composition containing at least one botanical active ingredient which increases the production by keratinocytes of at least one growth factor chosen from bFGF and PDGF.

This invention relates to a cosmetic skin care method intended to combat the cutaneous signs of ageing, including topical application to the skin of a botanical extract containing an active ingredient which increases the production of certain growth factors via keratinocytes.

The skin consists primarily of three layers, namely, starting with the most superficial one, the epidermis, the dermis and the hypoderm.

The epidermis, in particular, consists of keratinocytes (in majority), melanocytes (involved in the pigmentation of the skin) and Langerhans' cells. Its function is to protect the body from the outside environment and to ensure its integrity, and in particular to impede the penetration of micro-organisms and chemical substances, and prevent evaporation of the water contained in the skin, which could lead to dehydration.

The dermis provides a solid support to the epidermis and also ensures its nutrition. In particular, it consists of fibroblasts and an extracellular matrix consisting primarily of collagen, elastin and proteoglycans.

Collagen fibres, in particular, contribute to the firmness of the skin. They have a tendency to diminish with age, in particular after menopause, due to less natural renewal and stronger collagenase activity, which degrades them, resulting in a thinning of the dermis and leading to a slackening of the skin.

Such being the case, new active cosmetic ingredients are continuously being sought, which make it possible to fight against the cutaneous signs of ageing and, in particular, against atrophy of the dermis.

In this regard, it is known that certain plant growth factors, applied topically to the skin, are capable of exercising a cosmetic or dermatological activity, resulting in the attenuation of certain cutaneous signs of ageing.

Thus, the application FR-2 854 328 describes the use of plant growth factors of proteinaceous origin, such as phytosulfokine, in order to induce the differentiation and/or proliferation of cells such as fibroblasts, and to thereby combat cutaneous ageing, and in order to promote healing of the skin.

It is also known that these anti-ageing and healing effects can alternatively be obtained by applying compounds to the skin such as lipopolysaccharides, having an activity similar to that of growth factors such as the EGF (Epidermal Growth Factor) (EP-0 404 661), or else compounds promoting the production of growth factors such as VEGF (Vascular Endothelial Growth Factor), in particular, an extract of crocus (saffron) flowers and stamens, which improves vascularisation and thus certain symptoms of cutaneous ageing, such as the loss of radiant skin tone (JP2005-041811).

In this same connection, topical application of α-hydroxy acids has been used for many years for improving various cutaneous conditions and, in particular, for lessening the signs of photoageing. Such being the case, it is known that these compounds have the effect of increasing the secretion of cytokines such as the VEGF by the epidermis, and that this mechanism is probably responsible for the improved vascularisation observed with these compounds, thereby partially contributing to their anti-ageing effect (RENDL. M et al, British Journal of Dermatology 2001; 145: 3-9).

Finally, the applicant has demonstrated that two botanical extracts, a vanilla extract and an extracted oil of the Magnolia champaca flower, had, in particular, a synthesis stimulating activity for the PDGF growth factor and were able to be used in particular for preventing or treating cutaneous ageing.

However, the need still remains to propose new cosmetic active ingredients making it possible to more effectively combat the cutaneous signs of ageing and, in particular, the loss of firmness to the skin.

Furthermore, taking into account the ever-growing search by consumers for natural products containing the least number of synthetic ingredients possible, and increasingly heavy regulatory constraints besetting compounds coming from the chemical industry, it would seem desirable for these active cosmetic ingredients to be of plant origin.

Such being the case, the applicant deserved credit for showing that it was possible to act topically on a new biological target in order to combat the loss of firmness to the skin, for developing a screening test for selecting active ingredients acting on this target and for identifying numerous plant extracts responding to this test, thereby making it possible to meet the aforesaid needs.

Thus, the object of this invention is a cosmetic skin care method, intended to prevent and/or treat at least one sign of cutaneous ageing, including topical application to the skin of a composition containing at least one botanical active ingredient which increases the production by keratinocytes of at least one human growth factor chosen from: bFGF and PDGF, other than a vanilla extract or an oil extracted from the Magnolia champaca flower.

Another object of this invention is the cosmetic use of a botanical active ingredient which increases the production by keratinocytes of at least one human growth factor chosen from: bFGF and PDGF, other than a vanilla extract or an oil extracted from the Magnolia champaca flower, for preventing and/or treating at least one sign of cutaneous ageing.

As an introduction, it is specified that by “active ingredient which increases the production by keratinocytes of at least one growth factor,” is meant a compound or (in particular in the case of a botanical extract) a mixture of compounds capable of increasing the production of the aforesaid growth factors in comparison with an untreated control, determined, in particular, by means of the ELISA method, as described in the Examples below. Preferably, the difference between the increase in the quantity of growth factors produced by the tested extract, in comparison with the untreated control, and the standard deviation observed in the test will be at least 20%, better, at least 40%, even better, at least 60%, still better, at least 80% or even at least 100%. By “botanical active ingredient”, it is intended to designate a mixture of compounds extracted from a plant, not a single purified compound.

The fact of using compounds increasing the production of certain growth factors by keratinocytes makes it possible to take advantage of the secretory capabilities of the epidermis and to act on a target (the epidermis) that can be accessed more easily by cosmetic products than the dermis, in order to indirectly obtain a dermic effect with these products, after the growth factors have accumulated in the extra-cellular matrix and have induced the production of collagen by the fibroblasts. This mode of action of the active ingredients used according to the invention has the further advantage of satisfying the regulatory requirements for these cosmetic products, the sought-after dermic effect not being obtained by a direct action on the dermis, which is prohibited in cosmetics, but which rather comes under dermatology.

The growth factors aimed at in this invention are chosen from: bFGF (Basic Fibroblast Growth Factor) and PDGF (Platelet-Derived Growth Factor). It is preferred to use an active ingredient stimulating the production of bFGF or PDGF.

The active ingredient promoting the production of these growth factors can be used at the rate of 0.00001 to 10% by weight, preferably at the rate of 0.0001 to 5% by weight, and more preferably at the rate of 0.001 to 0.1% by weight, in relation to the total weight of the composition.

The active ingredients that can be used according to the invention are botanical extracts, i.e., active ingredients obtained by extraction, using any type of solvent, of any portion of a plant such as the bark, the wood, the rhizomes, the stems, the leaves or the flowers, for example. Examples of such active ingredients include extracts (of wood in particular) of Cedrus atlantica and extracts (of rhizomes in particular) of Zingiber cassumunar (bengle).

These extracts can be obtained according to a method including a step for extracting from these plants using an apolar organic solvent having a polarity index less than 1, such as hexane, cyclohexane, heptane and isooctane, possibly mixed with a polar organic solvent having a polarity index greater than 3.5, such as alcohol, in particular ethanol or isopropanol. This type of extraction is more particularly suited to the extraction of Zingiber cassumunar Roxb.

As an alternative, the active ingredient used according to the invention can be obtained according to a method including a step for extracting vapour distillation residues from the plant in question, after elimination of the essential oils, by using a polar organic solvent having a polarity index greater than 3.5, such as an alcohol, in particular methanol, ethanol or isopropanol, possibly mixed with an apolar organic solvent having a polarity index less than 1, such as those cited above. This extraction method is more particularly suited to the extraction of Cedrus atlantica.

In every case, the extraction can be carried out on all or part of the plant involved, which can be ground or broken into pieces in the usual manner. The extraction is generally carried out by immersing or gently stirring the ground material into one or more of the aforesaid solvents at temperatures ranging, for example, from ambient temperature to 100° C., for a time period of approximately 30 min. to 12 hrs. The solution is then preferably filtered so as to eliminate the insoluble substances from the plant. Where appropriate, the solvent is also eliminated, if it is a matter of a volatile solvent such ethanol, methanol, hexane or cyclohexane, for example.

This extraction step is common in the field of plant extracts, and those skilled in the art are capable of adjusting the reaction parameters thereof, based on their general knowledge.

The cutaneous signs of ageing aimed at in this invention can be chronological (intrinsic) or actinic (photo-ageing) signs of ageing. More particularly, the invention aims to prevent and/or treat the cutaneous signs linked to the slow-down in production and/or to the degradation of collagen, such as the formation of wrinkles and fine lines, the loss of firmness to the skin and/or dermic atrophy.

Preferably, the active ingredient used according to the invention, or the composition implemented in the method according to the invention, are applied to the human skin, in particular to wrinkled skin, more particularly to the skin of menopausal women. It can advantageously be applied to the skin of the face, neck or possibly the neckline or, as an alternative, to any part of the body.

The composition containing this active ingredient can be applied in the morning and/or in the evening, preferably in the evening, over the entire face, neck and possibly the neckline, or even the body.

Besides the previously described active ingredient, the composition implemented according to the invention generally includes a physiologically acceptable and preferably a cosmetically acceptable medium, i.e., which does not cause uncomfortable sensations (flushing, nagging pains, tingling sensations . . . ) which are unacceptable for the user.

This medium generally contains water and possibly other solvents such as ethanol.

The composition used according to the invention can be in any form suited to topical application to the skin and, in particular, in the form of an emulsion of oil-in-water, water-in-oil or multiple emulsions (W/O/W or O/W/O), which can possibly be microemulsions or nanoemulsions, or in the form of a hydrodispersion, solution, aqueous gel or powder. It is preferred that this composition be in the form of an oil-in-water emulsion.

This composition is preferably used as a care or cleaning product for the skin of the face and/or the body and, in particular, can be in the form of a fluid, gel or foam, packaged, for example, in a pump bottle, aerosol can or tube, or as a cream packaged, for example, in a jar. As an alternative, it can be in the form of a makeup product and, in particular, a foundation or a loose or compressed powder.

It can contain various additives, such as at least one compound chosen from:
oils, which can be chosen, in particular, from: volatile or non-volatile, linear or cyclic silicone oils, such as dimethylpolysiloxanes (dimethicones), polyalkylcyclosiloxanes (cyclomethicones) and polyalklyphenylsiloxanes (phenyldimethicones); synthetic oils such as fluorinated oils, alkyl benzoates and branched hydrocarbons such as polybutene; vegetable oils and, in particular, soybean or jojoba oil; and mineral oils such as paraffin oil;
waxes, such as ozocerite, polyethylene wax, beeswax or carnauba wax;
silicone elastomers obtained, in particular, by reacting, in the presence of a catalyst, a polysiloxane having at least one reactive group (hydrogen or vinyl, in particular) and carrying at least one end and/or side alkyl (in particular methyl) or phenyl group, with an organosilicon such as an organohydrogenpolysiloxane;
surfactants, preferably emulsifiers, whether non-ionic, anionic, cationic or amphoteric, and, in particular, esters of fatty acids and polyols, such as esters of fatty acids and glycerol, esters of fatty acids and sorbitan, esters of fatty acids and polyethylene glycol; esters of fatty acids and sucrose; esters of fatty alcohols and polyethylene glycol; alkylpolyglucosides; modified polysiloxanes polyethers; betaine and its derivatives; polyquaterniums; sulphate salts of ethoxylated fatty alcohols; sulfosuccinates; sarcosinates; alkyl- and dialkylphosphates and their salts; and soaps of fatty acids;
cosurfactants such as linear fatty alcohols and, in particular, hexadecyl and stearyl alcohols;
thickeners and/or gelling agents, and, in particular, hydrophilic or amphiphilic, crosslinked or non-crosslinked homo- and copolymers of acrylamidoethylpropane sulfonic acid (AMPS) and/or of acrylamide and/or of acrylic acid and/or of salts or esters of acrylic acid; xanthan or guar gum; cellulose derivatives; and silicone gums (dimethiconol);
humectants, such as polyols, including gylcerin, propylene glycol and sugars, and mucopolysaccharides such as hyaluronic acid and its salts and esters;
organic filters, such as derivatives of dibenzoylmethane (including butyl methoxydibenzoylmethane), derivatives of cinnamic acid (including ethylhexyl methoxycinnamate), salicylates, para-aminobenzoic acids, β-β′-diphenylacrylates, benzophenones, derivatives of benzylidene camphor, phenylbenzimidazoles, triazines, phenylbenzotriazoles and anthranilic derivatives;
mineral oxide-based in organic filters in the form of coated or uncoated pigments or nanopigments and, in particular, titanium dioxide or zinc oxide-based;
colorants;
preservatives;
fillers and, in particular, soft-focus powders, which can, in particular, be chosen from polyamides, silica, talc, mica, fibres (polyamide and cellulose, in particular);
tightening agents and, in particular, plant proteins, synthetic latexes (acrylic in particular) and colloidal dispersions of inorganic fillers;
sequestering agents such as the salts of EDTA;
fragrances;
and their mixtures, without this list being limiting.

Examples of such additives are cited in particular in the CTFA Dictionary (International Cosmetic Ingredient Dictionary and Handbook published by the Cosmetic, Toiletry and Fragrance Association, 9^{th} Edition, 2002).

The composition used according to the invention can further include active ingredients other than those promoting the production of the aforesaid growth factors, and in particular at least one active ingredient chosen from: agents stimulating the production of the growth factors TGF-α or β and/or HBEGF (Heparin-Binding Epidermal Growth Factor) and/or VEGF; anti-glycation or deglycating agents; agents increasing the synthesis of collagen or preventing its degradation (anti-collagenase agents, in particular matrix metalloproteinase inhibitors); agents increasing the synthesis of elastin or preventing its degradation (anti-elastase agents); agents increasing the synthesis of glycosaminoglycanes or proteoglycanes or preventing their degradation (anti-proteoglycanase agents); agents increasing the proliferation or differentiation of keratinocytes; agents increasing the proliferation of fibroblasts; depigmenting or anti-pigmenting agents; anti-oxidising or anti-radical or anti-pollution agents; agents increasing the synthesis of epidermic lipids; and their mixtures, without this list being limiting.

Examples of such agents are, in particular: plant extracts and, in particular, extracts of Chondrus crispus, Thermus thermophilus, Pisum sativum, Centella asiatica, Scenedesmus, Moringa pterygosperma, Witch-hazel, Castanea sativa, Hibiscus sabdriffa, Polyanthes tuberosa, Argania spinosa, Aloe vera, Narcissus tarzetta, or licorice; an essential oil of Citrus aurantium (Neroli); α-hydroxy acids such as glycolic, lactic and citric acids, and their esters; β-hydroxy acids, such as salicylic acid and its derivatives; hydrolyzates of plant proteins (in particular soybean or hazelnut); acylated oligopeptides (marketed in particular by the SEDERMA Company under the tradenames Maxilip®, Matrixyl® 3000, Biopeptide® CL or Biopeptide® EL); yeast extracts and, in particular, Saccharomyces cerevisiae; algae extracts and, in particular, sea cabbages; vitamins and their derivatives such as retinyl palmitate, ascorbic acid, ascorbyl glucoside, magnesium or sodium phosphate ascorbyl, ascorbyl palmitate, ascorbyl tetraisopalmitate, ascorbyl sorbate, tocopherol, tocopheryl acetate and tocopheryl sorbate; homo- and copolymers of methacryloyloxyethylphosphorylcholine; urea; ceramides and phospholipids; arbutin; kojic acid; ellagic acid; and their mixtures.

The invention will now illustrated by the following non-limiting examples.

### EXAMPLES

### Example 1

### Test for Increasing the Production of bFGF

### Extracts Tested:

The activity of a botanical extract was evaluated, namely an extract of Cedrus atlantica, obtained by: vapour distillation of cedar wood, elimination of the essential oil obtained, recovery of the distillation and extraction residues with a hexane/isopropanol mixture, filtration, recovery of the filtrate and evaporation of the mixture of solvents, take-up via dipropylene glycol and filtration in order to obtain a viscous liquid extract.

### Protocol:

The activity of the botanical extract with respect to the production of bFGF was measured by quantitative evaluation of the concentrations of the human bFGF growth factor in keratinocyte cultures, by means of the ELISA method, by using the Quantikine® immunoassay kit (No. DFB50, R&D Systems).

The keratinocyte cultures were prepared as follows: keratinocytes derived from neonatal foreskins (Cambrex or Cascade Biologics) previously grown for multiplication in culture medium suited to the growth of keratinocytes (KGM Bullet Kit, Cambrex) were seeded in 6-well plates.

After 24 hours of culture in an oven at 37° C. with 5% CO₂ and at moisture saturation, the confluent cells were washed with PBS (Gibco) buffer at 7.4 pH and incubated with alkaline-specific medium (KGM, Cambrex) containing the product being tested, for 24 hours, at the concentrations provided herein below. The product was tested in triplicate for two donors. A control with no product being tested (so-called “untreated”) was also produced while keeping the cells in the same medium, without treatment.

### Results:

TABLE 1

| | | Stimulation of bFGF (%) | |
|---|---|---|---|
| | | | Standard |
| Extract tested | Concentration | Average (%)* | deviation |
| Cedrus atlantica | 0.0008% | 143.3 | 21 |

*in relation to the untreated control

It follows from this test that the Cedrus atlantica extract used makes it possible to increase the production of bFGF. Such being the case, it is known that this growth factor in particular has the capability of inducing the proliferation of the fibroblasts and the migration of the keratinocytes (Ashcroft GS et al., J. Anat. 1997, 180 (Pt. 3): 351-65). Thus, it appears that, via its effect of increasing the production of bFGF, the extract tested may make it possible to combat cutaneous ageing by being applied topically to the skin.

### Example 2

### Test for Increasing the Production of PDGF

### Protocol:

In a manner similar to Example 1, the activity of a Bengle (Zingiber cassumunar Roxb.) extract was evaluated in relation to the production of PDGF, via quantitative evaluation of the concentrations of the human PDGF-AA growth factor in cell cultures, by means of the ELISA method, by using the Quantikine® immunoassay kit (No. DAA00, R&D Systems) and keratinocyte culture conditions identical to those of Example 1.

This extract was obtained via extraction of dried bengle roots using a (80/20) mixture of hexane and isopropyl alcohol, followed by filtration and then vacuum evaporation of the solvent present in the filtrate, and finally molecular distillation of the oleoresin obtained.

The molecular distillation process consisted in distilling the oleoresin via passage into a molecular distillation device of the KDL4 type (UIC GmH), according to the parameters given in Table 2 below.

TABLE 2

| Distillation parameters | |
|---|---|
| Rate of insertion (ml/hr) | 400 |
| Evaporator temperature (° C.) | 125 |
| Condenser temperature (° C.) | 65 |
| Product introduction temperature (° C.) | 70 |
| Stirring (revolutions/min) | 200 |
| Vacuum pressure (mbar) | 9.5 · 10⁻² |

Next, the still bottoms were recovered and then subjected to washing with ethanol at 96.2° and with activated carbon, at a temperature of 50-60° C., for the purpose of bleaching them. The filtrate thus obtained was subjected to a second washing operation under the same conditions. The final filtrate was then filtered on a conical filter in order to eliminate the activated carbon residues, and then the ethanol was vacuum-evaporated.

Results: tested at 10 μg/ml (0.001%), the bangle extract increases the synthesis of PDGF by an average of 148% (in relation to the untreated control) ±20%.

In as much as it was demonstrated that the rate of PDGF diminished with age (Karlsson C. et al., J. Cell. Physiol., 1994, 158 (2): 256-62), it is thus possible to apply this PDGF synthesis activator topically to the skin in order to combat the cutaneous signs of ageing.

### Example 3

### Stimulation of Fibroblast Proliferation by PDGF and FGFb

### Method:

Human dermal fibroblasts from a single donor were obtained from Cascade. At the 7^{rd} passage, cells were seeded in 96 well plates and cultured in Dulbecco's modified Eagle medium DMEM (Gibco BRL, Gaithersburg, USA) supplemented with 10% foetal bovine serum (FBS, PAA, Linz, Austria), 25 mm L-glutamine (Gibco) and 1% penicillin/streptomycin (Gibco). All cell culture was performed at 7° C. in 5% CO₂ and 95% air.
This fibroblast culture was incubated by two growth factors (BFGF and FGFb) at various concentrations for 24 h. After 24 hours incubation, 200 μL dosed media from 96-well plate were removed. CellTiter 96 Aqueous One Solution Reagent was used as described by the manufacturer. The absorbance at 490 nm was recorded using a plate reader to evaluate cell proliferation. The controls were the non treated cells.
The experiment was conducted in 8 times by concentration.

### Results:

The results of the stimulation of fibroblast proliferation compared to the control are given in Table 3 below.

TABLE 3

| | Stimulation (%) | SD (%) |
|---|---|---|
| Untreated | 100.00 | 10.39 |
| PDGF 1 ng/ml | 107.20 | 10.98 |
| PDGF 10 ng/ml | 154.37 | 15.94 |
| PDGF 30 ng/ml | 185.58 | 8.14 |
| FGFb (157 aa) 1 ng/ml | 141.22 | 23.23 |
| FGFb (157 aa) 10 ng/ml | 149.20 | 15.00 |

From this experiment, it appears that the growth factors tested significantly enhanced fibroblast proliferation. Active agents which stimulate the production of these growth factors in the epidermis will thus result in an increase in collagen synthesis and thus provide for a dermal anti-ageing effect of the cosmetic compositions containing these agents.

### Example 4

### Oil-In-Water Emulsions (O/W)

The following compositions can be prepared conventionally for those skilled in the art. The quantities indicated below are expressed in weight percents. The ingredients in uppercase letters are identified in accordance with the INCI nomenclature.

Emulsion A

| CETEARYL ALCOHOL & CETEARYL GLUCOSIDE | 4.00% |
|---|---|
| BEHENETH-25 | 2.00% |
| Cedrus atlantica extract* | 1.00% |
| Licorice extract | 0.10% |
| Emollients | 35.00% |
| Tocopheryl acetate | 0.50% |
| DIMETHICONE | 2.00% |
| EDTA | 0.05% |
| Glycerin | 5.00% |
| Gelling agents | 2.00% |
| pH adjuster | q.s.f. |
| Preservatives | q.s.f. |
| Water | q.s.f. 100.00% |
| *prepared as described in Example 1 | |

Emulsion B

| Bengle extract* | 0.01% |
|---|---|
| METHYLSILANOL MANNURONATE | 5.00% |
| STEARETH-21 | 1.50% |
| Tocopheryl acetate | 0.50% |
| Vegetable oils | 5.00% |
| Silicon oils | 6.00% |
| Octyldodecanol | 2.00% |
| Glycerin | 5.00% |
| Gelling agents | 2.80% |
| Denatured alcohol | 5.00% |
| Sequestering agent | 0.05% |
| pH adjuster | q.s.f. |
| Preservatives | q.s.f. |
| Water | q.s.f. 100.00% |
| *prepared as described in Example 2 | |

1-15. (canceled)

16. A cosmetic skin care method for preventing and/or treating at least one sign of cutaneous ageing, including topical application to the skin of a composition containing at least one botanical active ingredient which increases the production by keratinocytes of at least one human growth factor chosen from: bFGF and PDGF, other than a vanilla extract or an oil extracted from the Magnolia champaca flower.

17. The method of claim 16, wherein said active ingredient a Cedrus atlantica extract.

18. The method of claim 16, wherein said active ingredient is obtained according to a method including a step for extracting vapour distillation residues, after elimination of the essential oils, by using a polar organic solvent having a polarity index greater than 3.5, possibly mixed with an apolar organic solvent having a polarity index less than 1.

19. The method of claim 16, wherein the growth factor is the bFGF.

20. The method of claim 18, wherein said polar organic solvent is chosen from alcohols such as ethanol and isopropanol.

21. The method of claim 18, wherein said apolar organic solvent is chosen from: hexane, cyclohexane, heptane and isooctane.

22. The method of claims 16, wherein said sign is linked to the slow-down in the production, and/or to the degradation of collagen, such as the formation of wrinkles and fine lines, the loss of firmness to the skin and/or dermic atrophy.

23. The method of claim 16, wherein the composition is in the form of an oil-in-water emulsion.

24. A cosmetic skin care method for preventing and/or treating at least one sign of cutaneous ageing, including topical application to the skin of a composition containing at least a Zingiber cassumunar Roxb extract which increases the production by keratinocytes of at least one human growth factor chosen from: bFGF and PDGF.

25. The method of claim 24, wherein said Zingiber cassumunar Roxb extract is obtained according to a method including an extraction step using an apolar organic solvent having a polarity index less that 1, possibly mixed with a polar organic solvent having a polarity index greater than 3.5.

26. The method of claim 24, wherein the growth factor is the PDGF.

27. The method of claim 25, wherein said polar organic solvent is chosen from alcohols such as ethanol and isopropanol.

28. The method of claim 25, wherein said apolar organic solvent is chosen from: hexane, cyclohexane, heptane and isooctane.

29. The method of claim 24, wherein said sign is linked to the slow-down in the production, and/or to the degradation of collagen, such as the formation of wrinkles and fine lines, the loss of firmness to the skin and/or dermic atrophy.

30. The method of claim 24, wherein the composition is in the form of an oil-in-water emulsion.
